(19) United States
(12) Patent Application Publication
(10) Pub. No.: US 2022/0313156 A1
(43) Pub. Date: Oct. 6, 2022

(54) MONITORING SYSTEM AND MONITORING METHOD FOR SLEEP APNEA

(71) Applicant: Osense Technology Co., Ltd., Taipei City (TW)

(72) Inventors: FU-KUEI CHEN, Taipei City (TW); YOU-KWANG WANG, Taipei City (TW); HSIN-PIAO LIN, Taoyuan City (TW)

(73) Assignee: Osense Technology Co., Ltd., Taipei City (TW)

(21) Appl. No.: 17/378,797

(22) Filed: Jul. 19, 2021

Related U.S. Application Data
(60) Provisional application No. 63/171,091, filed on Apr. 6, 2021.

(30) Foreign Application Priority Data
Jun. 16, 2021 (TW) 110121917

(51) Int. Cl. A61B 5/00; A61B 5/0205; G16H 50/30

(52) U.S. Cl. CPC A61B 5/4818; A61B 5/0205; A61B 5/7257; A61B 5/7225; A61B 5/7267; G16H 50/30

(57) ABSTRACT

A monitoring system and a monitoring method for sleep apnea are provided. The monitoring method includes: obtaining a regression model; transmitting a radio frequency (RF) signal to a subject and receiving a reflection signal corresponding to the RF signal, where the reflection signal includes a heartbeat signal, a respiration signal, and a movement signal;

performing wavelet entropy analysis on the heartbeat signal and the respiration signal respectively to generate a first entropy corresponding to the heartbeat signal and a second entropy corresponding to the respiration signal; calculating, based on the regression model, an apnea hypopnea index (AHI) according to the first entropy, the second entropy, and the movement signal; determining whether a sleep apnea event occurs on the subject according to the AHI, so as to generate a determination result; and outputting the determination result.

### CROSS-REFERENCE TO RELATED APPLICATION

This application claims the priority benefit of U.S. provisional application Ser. No. 63/171,091, filed on Apr. 6, 2021 and Taiwan application serial no. 110121917, filed on Jun. 16, 2021. The entirety of each of the above-mentioned patent applications is hereby incorporated by reference herein and made a part of this specification.

### BACKGROUND

### Technical Field

The disclosure generally relates to a monitoring system and a monitoring method for sleep apnea.

### Description of Related Art

When sleep apnea patients sleep, recurrent collapse of the upper respiratory tract (including the nasopharynx, oropharynx, and larynx) of the patients causes obstruction to their respiratory tract. Their breathing becomes shallow and takes more efforts. In the case of severe symptoms, the patients may be unable to breathe and suffocate. In most patients, obesity leads to a narrow respiratory tract or insufficient airway muscle tone, which causes collapse of their upper respiratory tract. In addition, some of the patients have a narrow respiratory tract due to factors such as narrower or receding chins, large tonsils or palatine uvula, or congenital craniofacial anomalies.

Sleep apnea patients are subject to drowsiness during daytime and have a hard time concentrating. As a result, their working efficiency reduces, and accidents could even happen, for example, when driving under influence of drowsiness. Furthermore, sleep apnea patients may develop angina pectoris, myocardial infarction, or stroke during a sleep. Moreover, sleep apnea patients may develop sudden memory loss or early-onset dementia. On the other hand, their personalities may change due to sleep apnea (such as anxiety, sleep deprivation, bad temper, or restlessness), which may even lead to depression or insomnia of the patients.

Accordingly, how to detect whether people suffer from sleep apnea at an early stage has emerged as an issue in the art.

### SUMMARY

The disclosure is directed to a monitoring system and a monitoring method for sleep apnea which can monitor a sleep state of a subject.

A monitoring system for sleep apnea of the disclosure is adapted to monitor a subject. The monitoring system includes a processor, a storage medium, and a transceiver. The storage medium stores a regression model. The processor is coupled to the storage medium and the transceiver. The processor is configured to execute the following. A radio frequency signal is transmitted to the subject through the transceiver, and a reflection signal corresponding to the radio frequency signal is received. The reflection signal includes a heartbeat signal, a respiration signal, and a movement signal. Wavelet entropy analysis is respectively performed on the heartbeat signal and the respiration signal to generate a first entropy corresponding to the heartbeat signal and a second entropy corresponding to the respiration signal. An apnea hypopnea index (AHI) is calculated based on the regression model according to the first entropy, the second entropy, and the movement signal. It is determined whether a sleep apnea event occurs on the subject according to the AHI so as to generate a determination result. The determination result is output through the processor.

In an embodiment of the disclosure, the processor determines a movement number of the subject according to the movement signal and inputs the first entropy, the second entropy, and the movement number into the regression model to calculate the AHI.

In an embodiment of the disclosure, in response to the AHI being greater than a threshold value, the processor determines that the sleep apnea event occurs to generate the determination result.

In an embodiment of the disclosure, the storage medium further stores physiological information of the subject. In response to the AHI being less than or equal to a threshold value, the processor determines whether the sleep apnea event occurs according to the physiological information.

In an embodiment of the disclosure, the storage medium further stores a lookup table. The processor obtains a lookup value corresponding to the physiological information from the lookup table to generate the determination result.

In an embodiment of the disclosure, the physiological information includes at least one of a gender, an age, a height, a weight, and a neck circumference.

In an embodiment of the disclosure, the processor executes a fast Fourier transform on the reflection signal to generate a frequency spectrum. The processor executes first bandpass filtering on the frequency spectrum to generate the respiration signal and executes second bandpass filtering on the frequency spectrum to generate the heartbeat signal.

In an embodiment of the disclosure, a distance from the transceiver to the subject is between 0.5 m and 2 m.

In an embodiment of the disclosure, the processor receives training data through the transceiver and polysomnography (PSG) and trains the regression model according to the training data. The training data includes an examination result associated with polysomnography (PSG).

A monitoring method for sleep apnea of the disclosure is adapted to monitor a subject. The monitoring method includes the following. A regression model is obtained. A radio frequency signal is transmitted to the subject, and a reflection signal corresponding to the radio frequency signal is received. The reflection signal includes a heartbeat signal, a respiration signal, and a movement signal. Wavelet entropy analysis is respectively performed on the heartbeat signal and the respiration signal to generate a first entropy corresponding to the heartbeat signal and a second entropy corresponding to the respiration signal. An AHI is calculated based on the regression model according to the first entropy, the second entropy, and the movement signal. It is determined whether a sleep apnea event occurs on the subject according to the AHI so as to generate a determination result. The determination result is output.

Based on the above, without requiring a subject to wear any wearable devices, the monitoring system of the disclosure can measure a sleep state of the subject in a non-contact way and generate a determination result indicating whether a sleep apnea event occurs on the subject according to the sleep state.

### BRIEF DESCRIPTION OF THE DRAWINGS

FIG. 1 is a schematic diagram of a monitoring system for sleep apnea according to an embodiment of the disclosure.

FIG. 2 is a flowchart of a monitoring method for sleep apnea according to an embodiment of the disclosure.

FIG. 3 is a schematic diagram of a frequency spectrum of a reflection signal according to an embodiment of the disclosure.

FIG. 4 is a flowchart of a monitoring method for sleep apnea according to another embodiment of the disclosure.

### DESCRIPTION OF THE EMBODIMENTS

To make the disclosure more comprehensible, embodiments accompanied with drawings are described in detail below. Wherever possible, the same reference numbers of the elements/components/steps are used in the drawings and the description to refer to the same or like parts.

To measure a sleep state of a subject, the subject usually has to stay at a sleep center overnight to receive polysomnography (PSG) during a sleep. Polysomnography may be performed to obtain information of the subject such as sleep brainwaves, an electrooculogram, an electromyogram, an electrocardiogram, a number of snores, nasal and oral breathing flow rates, chest and abdominal breathing movements, an oxygen saturation index, an apnea hypopnea index (AHI), body movements, or sleep postures. Health professionals may determine whether the subject is prone to a risk of developing sleep apnea according to a result of polysomnography. However, the method above is costly and time-consuming and requires well-trained professionals to conduct. Therefore, it is hard for the method above to be applied widely.

Accordingly, the disclosure provides a monitoring system used at home which can measure a sleep state of a subject in a non-contact way to determine whether the subject is at a risk of developing sleep apnea. Therefore, the subject does not have to spend money and time going to the sleep center nor wear any wearable devices which would affect the sleep quality.

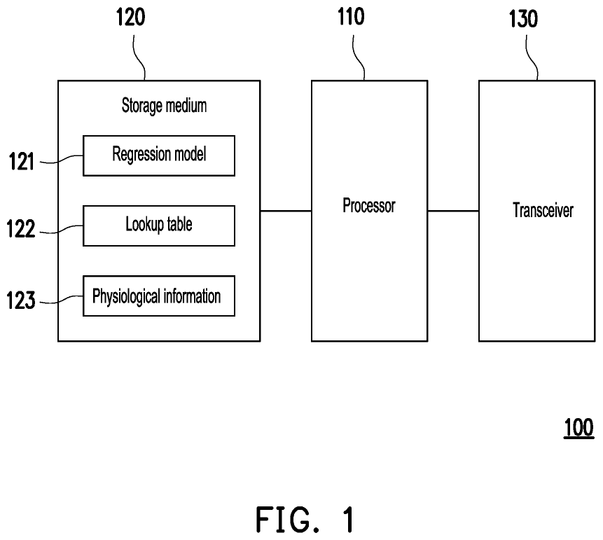

FIG. 1 is a schematic diagram of a monitoring system 100 for sleep apnea according to an embodiment of the disclosure. The monitoring system 100 is adapted to monitor a sleep state of a subject. The monitoring system 100 may include a processor 110, a storage medium 120, and a transceiver 130.

The processor 110 is, for example, a central processing unit (CPU) or other programmable general-purpose or special-purpose micro control unit (MCU), microprocessor, digital signal processor (DSP), programmable controller, application specific integrated circuit (ASIC), graphics processing unit (GPU), image signal processor (ISP), image processing unit (IPU), arithmetic logic unit (ALU), complex programmable logic device (CPLD), field programmable gate array (FPGA), or other similar elements or combinations of the above elements. The processor 110 may be coupled to the storage medium 120 and the transceiver 130 and access and execute multiple modules and various applications stored in the storage medium 120.

The storage medium 120 is, for example, any type of fixed or movable random access memory (RAM), read-only memory (ROM), flash memory, hard disk drive (HDD), solid state drive (SSD) or similar elements or combinations thereof. The storage medium 120 is configured to store the multiple modules and various applications which may be executed by the processor 110.

The transceiver 130 transmits and receives a signal in a wireless manner. The transceiver 130 may further execute, for example, low noise amplification, impedance matching, frequency mixing, up or down frequency conversion, wave filtering, amplification, and similar operations. The transceiver 130 is, for example, a Doppler radar. The monitoring system 100 may emit a radio frequency signal such as a millimeter wave (mmWave) through the transceiver 130.

The storage medium 120 may store a regression model 121. The regression model 121 may be configured to determine the AHI corresponding to a physiological signal. In an embodiment, the processor 110 may train the regression model 121 and store the trained regression model 121 into the storage medium 120. Specifically, the processor 110 may receive training data through the transceiver 130 and polysomnography (PSG). The training data may include an examination result associated with polysomnography (PSG). The examination result may include information of the subject such as sleep brainwaves, an electrooculogram, an electromyogram, an electrocardiogram (i.e. a heartbeat signal), a number of snores, nasal and oral breathing flow rates, chest and abdominal breathing movements, a respiration signal corresponding to the nasal and oral breathing flow rates and/or the chest and abdominal breathing movements, an oxygen saturation index, an apnea hypopnea index (AHI), body movements, sleep postures, or a movement signal corresponding to the body movements and/or the sleep postures. The processor 110 may perform multiple regression analysis on the training data to generate the regression model 121 corresponding to an entropy, a movement number, and the AHI. In other words, the regression model 121 includes at least information of three dimensions including the entropy, the movement number, and the AHI.

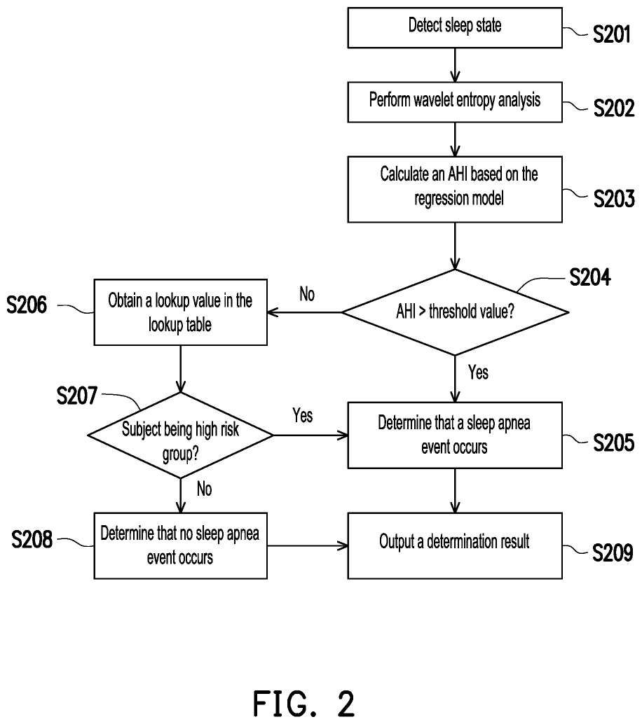

FIG. 2 is a flowchart of a monitoring method for sleep apnea according to an embodiment of the disclosure. The monitoring method is adapted to monitor a sleep state of a subject and may be performed by the monitoring system 100 shown in FIG. 1.

In step S201, the monitoring system 100 may detect a sleep state of a subject. Specifically, the processor 110 may transmit a radio frequency (RF) signal to the subject through the transceiver 130. The body of the subject reflects the radio frequency signal back to the transceiver 130. The processor 110 may receive a reflection signal corresponding to the radio frequency signal through the transceiver 130. The reflection signal may include a heartbeat signal, a respiration signal, and a movement signal. In an embodiment, a distance from the transceiver 130 to the subject may be approximately between 0.5 m and 2 m.

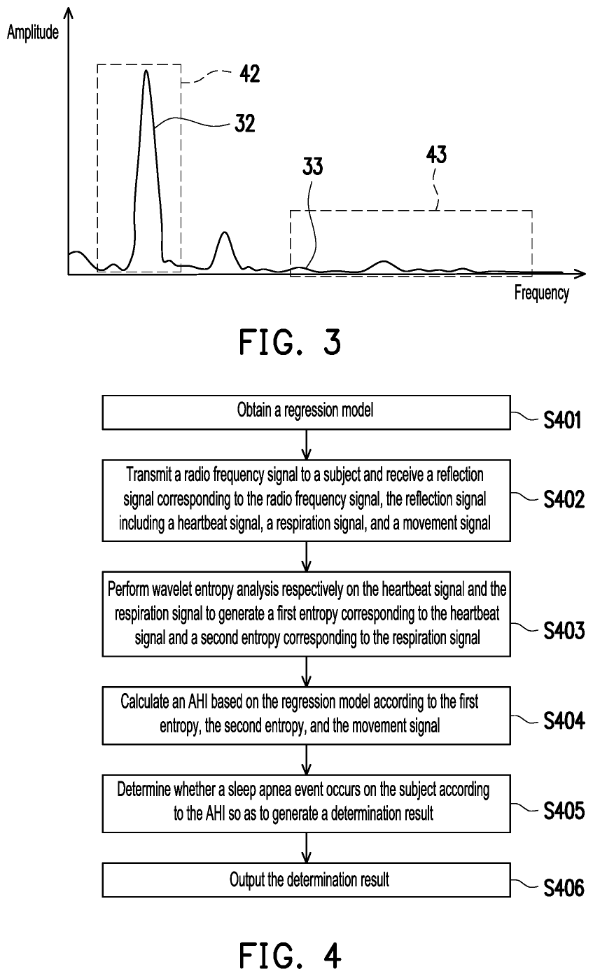

The processor 110 may perform signal processing on the reflection signal to obtain the heartbeat signal, the respiration signal, and the movement signal. In an embodiment, the processor 110 may determine a distance from the subject to the transceiver 130 according to the reflection signal so as to obtain the movement signal according to a change in the distance. In an embodiment, the processor 110 may obtain the heartbeat signal or the respiration signal from a frequency spectrum of the reflection signal. Specifically, the processor 110 may execute a fast Fourier transform (FFT) on the reflection signal to generate the frequency spectrum. FIG. 3 is a schematic diagram of a frequency spectrum of a reflection signal according to an embodiment of the disclosure. The frequency spectrum may include a peak value 32 corresponding to the respiration signal (or its harmonic wave) and a peak value 33 corresponding to the heartbeat signal (or its harmonic wave). A frequency corresponding to the peak value 33 is higher than a frequency corresponding to the peak value 32.

To retrieve the respiration signal and the heartbeat signal from the frequency spectrum, the processor 110 may use wave filters with different frequency bands to filter the frequency spectrum of the reflection signal. For example, the processor 110 may execute first bandpass filtering on the frequency spectrum to generate the respiration signal and execute second bandpass filtering on the frequency spectrum to generate the heartbeat signal. The first bandpass filtering may correspond to a window function 42, and the second bandpass filtering may correspond to a window function 43. A frequency band at which the window function 43 exists may be higher than a frequency band at which the window function 42 exists.

Returning back to FIG. 2, in step S202, the processor 110 may perform wavelet entropy analysis respectively on the heartbeat signal and the respiration signal to generate a first entropy corresponding to the heartbeat signal and a second entropy corresponding to the respiration signal.

In step S203, the processor 110 may calculate an AHI based on the regression model 121 according to the first entropy, the second entropy, and the movement signal. Specifically, the processor 110 determines a movement number (e.g. a number of body turns) of the subject according to the movement signal. After the movement number is obtained, the processor 110 may input the first entropy, the second entropy, and the movement number into the regression model 121 to calculate the AHI. In the following steps, the processor 110 may determine whether a sleep apnea event occurs on the subject according to the AHI to generate a determination result.

In step S204, the processor 110 may determine whether the AHI is greater than a threshold value. If the AHI is greater than the threshold value, the process proceeds to step S205. If the AHI is less than or equal to the threshold value, the process proceeds to step S206.

In step S205, the processor 110 may determine that a sleep apnea event occurs and generate a determination result. The determination result may indicate that a sleep apnea event has occurred on the subject during the sleep.

In an embodiment, the processor 110 may determine a severity of the sleep apnea event according to multiple threshold values and generate the corresponding determination result. The determination result may indicate whether the sleep apnea event occurring on the subject is slight, moderate, or severe. For example, the processor 110 may determine the severity of the sleep apnea event according to three threshold values including a first threshold value, a second threshold value, and a third threshold value. The first threshold value may be 5, the second threshold value may be 15, and the third threshold value may be 30. Each of the three threshold values is associated with an average value of the AHI in an hour. If an average AHI of the subject in an hour is less than the first threshold value, the processor 110 may determine that no sleep apnea event occurs. If the average AHI of the subject in an hour is less than the second threshold value and greater than or equal to the first threshold value, the processor 110 may determine that a slight sleep apnea event has occurred on the subject. If the average AHI of the subject in an hour is less than the third threshold value and greater than or equal to the second threshold value, the processor 110 may determine that a moderate sleep apnea event has occurred on the subject. If the average AHI of the subject in an hour is greater than or equal to the third threshold value, the processor 110 may determine that a severe sleep apnea event has occurred on the subject.

In step S206, the processor 110 may obtain, from a lookup table 122, a lookup value corresponding to physiological information 123 of the subject and the AHI. The lookup value indicates whether the subject is in a high risk group of developing sleep apnea. Specifically, the storage medium 120 may store the physiological information 123 of the subject and the lookup table 122 in advance. The physiological information 123 may include but not limited to information such as a gender, an age, a height, a weight, a neck circumference, etc. The physiological information 123 or the lookup table 122 is obtained, for example, by the processor 110.

In step S207, the processor 110 may determine whether the subject is in a high risk group of developing sleep apnea according to the lookup value. If the processor 110 determines that the subject is in the high risk group of developing sleep apnea, the process proceeds to step S205. If the subject is not in the high risk group of developing sleep apnea, the process proceeds to step S208.

Specifically, the lookup table 122 may record a mapping relation between information such as the physiological information 123 and the lookup value. In an embodiment, the lookup table 122 may record a mapping relation between the subject's age and the lookup value. The lookup value may indicate whether the subject is in the high risk group of developing sleep apnea. For example, the lookup table 122 may record that “a ratio of weight to height greater than 0.45, an age over 65, and a ratio of neck circumference to height greater than 0.24” correspond to a lookup value representing the high risk group. If the physiological information of the subject matches “a ratio of weight to height greater than 0.45, an age over 65, a ratio of neck circumference to height greater than 0.24”, the processor 110 may determine that the subject is in the high risk group of developing sleep apnea according to the corresponding lookup value.

In step S208, the processor 110 may determine that no sleep apnea event occurs and generate the determination result. The determination result may indicate that no sleep apnea event occurs on the subject during the sleep.

In step S209, the processor 110 may output the determination result for health professionals' reference.

FIG. 4 is a flowchart of a monitoring method for sleep apnea according to another embodiment of the disclosure. The monitoring method may be performed by the monitoring system 100 shown in FIG. 1. In step S401, a regression model is obtained. In step S402, a radio frequency (RF) signal is transmitted to a subject, and a reflection signal corresponding to the radio frequency signal is received. The reflection signal includes a heartbeat signal, a respiration signal, and a movement signal. In step S403, wavelet entropy analysis is respectively performed on the heartbeat signal and the respiration signal to generate a first entropy corresponding to the heartbeat signal and a second entropy corresponding to the respiration signal. In step S404, an AHI is calculated based on the regression model according to the first entropy, the second entropy, and the movement signal. In step 405, it is determined whether a sleep apnea event occurs on the subject according to the AHI so as to generate a determination result. In step S406, the determination result is output.

In summary of the above, the monitoring system of the disclosure may detect a sleep state of a subject by using a wireless signal. The monitoring system uses different wave filters to process a signal representing the sleep state of the subject to obtain information such as a heartbeat signal, a respiration signal, and a movement signal. The monitoring system further calculates an AHI of the subject according to a regression model and the information above. The AHI represents a severity of sleep apnea occurring on the subject. If the AHI is too high, the monitoring system determines that a sleep apnea event occurs on the subject during a sleep and generates a determination result. The monitoring system outputs the determination result. The determination result of the monitoring system assists health professionals in diagnosing whether the subject suffers from sleep apnea.

What is claimed is:

1. A monitoring system for sleep apnea adapted to monitor a subject, the monitoring system comprising:
a transceiver;
a storage medium storing a regression model; and
a processor coupled to the storage medium and the transceiver, wherein the processor is configured to:
transmit a radio frequency signal to the subject through the transceiver and receive a reflection signal corresponding to the radio frequency signal, wherein the reflection signal comprises a heartbeat signal, a respiration signal, and a movement signal;
perform wavelet entropy analysis respectively on the heartbeat signal and the respiration signal to generate a first entropy corresponding to the heartbeat signal and a second entropy corresponding to the respiration signal;
calculate an apnea hypopnea index based on the regression model according to the first entropy, the second entropy, and the movement signal;
determine whether a sleep apnea event occurs on the subject according to the apnea hypopnea index so as to generate a determination result; and
output the determination result.

2. The monitoring system according to claim 1, wherein the processor determines a movement number of the subject according to the movement signal and inputs the first entropy, the second entropy, and the movement number into the regression model to calculate the apnea hypopnea index.

3. The monitoring system according to claim 1, wherein in response to the apnea hypopnea index being greater than a threshold value, the processor determines that the sleep apnea event occurs to generate the determination result.

4. The monitoring system according to claim 1, wherein the storage medium further stores physiological information of the subject, wherein in response to the apnea hypopnea index being less than or equal to a threshold value, the processor determines whether the sleep apnea event occurs according to the physiological information.

5. The monitoring system according to claim 4, wherein the storage medium further stores a lookup table, wherein the processor obtains a lookup value corresponding to the physiological information from the lookup table to generate the determination result.

6. The monitoring system according to claim 4, wherein the physiological information comprises at least one of a gender, an age, a height, a weight, and a neck circumference.

7. The monitoring system according to claim 1, wherein the processor executes a fast Fourier transform on the reflection signal to generate a frequency spectrum, wherein the processor executes first bandpass filtering on the frequency spectrum to generate the respiration signal and executes second bandpass filtering on the frequency spectrum to generate the heartbeat signal.

8. The monitoring system according to claim 1, wherein a distance from the transceiver to the subject is approximately between 0.5 m and 2 m.

9. The monitoring system according to claim 1, wherein the processor receives training data through the transceiver and polysomnography(PSG) and trains the regression model according to the training data, wherein the training data comprises an examination result associated with polysomnography.

10. A monitoring method for sleep apnea adapted to monitor a subject, the monitoring method comprising:
obtaining a regression model;
transmitting a radio frequency signal to the subject and receiving a reflection signal corresponding to the radio frequency signal, wherein the reflection signal comprises a heartbeat signal, a respiration signal, and a movement signal;
performing wavelet entropy analysis respectively on the heartbeat signal and the respiration signal to generate a first entropy corresponding to the heartbeat signal and a second entropy corresponding to the respiration signal;
calculating an apnea hypopnea index based on the regression model according to the first entropy, the second entropy, and the movement signal;
determining whether a sleep apnea event occurs on the subject according to the apnea hypopnea index so as to generate a determination result; and
outputting the determination result.
